(19) United States
(12) Patent Application Publication
(10) Pub. No.: US 2020/0069575 A1
(43) Pub. Date: Mar. 5, 2020

(54) OPHTHALMIC FORMULATIONS, PROCESS FOR PREPARING THE SAME AND METHOD FOR ADMINISTERING THE SAME

(71) Applicants: Yunxiang LIU, San Diego, CA (US); Eric LIU, Medford, MA (US)

(72) Inventors: Yunxiang LIU, San Diego, CA (US); Eric LIU, Medford, MA (US)

(21) Appl. No.: 16/554,134

(22) Filed: Aug. 28, 2019

Related U.S. Application Data
(60) Provisional application No. 62/725,110, filed on Aug. 30, 2018.

(51) Int. Cl. A61K 9/00; A61K 31/137; A61K 47/34; A61K 31/222

(52) U.S. Cl. CPC A61K 9/0024; A61K 9/0051; A61K 31/222; A61K 47/34; A61K 31/137

(57) ABSTRACT

Disclosed are ophthalmic formulations, processes for preparing the same and methods for treating and preventing oculopathy with the same.

### CROSS-REFERENCE TO RELATED APPLICATIONS

The present application claims priority to and the benefit of U.S. Provisional Application No. 62/725,110, filed Aug. 30, 2018, the disclosure of which is hereby incorporated by reference in its entirety.

### FIELD

The present disclosure generally relates to the medical field. In particular, the present disclosure relates to the ophthalmic field.

### BACKGROUND

Eyelids consist of thin folds of skin, muscle, and connective tissue. The eyelids protect the eyes and spread tears over the front of the eyes. The inside of the eyelids are lined with the conjunctiva of the eyelid (the palpebral conjunctiva), and the outside of the lids are covered with the body's thinnest skin. Some common eyelid disorders include the following: stye, blepharitis, chalazion, entropion, ectropion, eyelid edema, eyelid tumors and myasthenia gravis.

The main treatment for eyelid disorders is currently by administration oral preparation or eyedrops. However, unwanted systemic side effects can often occur with administration oral preparation, including nausea/vomiting, diarrhea, stomach pain, increased salivation and tearing, irregular heartbeat, restlessness, anxiety, muscle twitching or tremor, blurred vision, and difficulty breathing. In addition, dosing with oral preparation or eyedrops is multiple times a day, which can negatively impact quality of life and reduce compliance.

### SUMMARY

In one aspect, the present disclosure relates to an ophthalmic mesh-like formulation, comprising an active pharmaceutical ingredient, a biodegradable material and a pharmaceutically acceptable excipient, wherein the mesh-like formulation has a thickness of not more than 2 mm, a long diameter of not more than 22 mm, and a short diameter of not more than 4 mm.

In another aspect, the present disclosure relates to an ophthalmic mesh-like formulation, comprising an active pharmaceutical ingredient and a biodegradable material, wherein the biodegradable material comprises PLGA (poly(lactic-co-glycolic acid)) and PLA (polylactic acid).

In yet another aspect, the present disclosure relates to an ophthalmic mesh-like formulation, comprising an active pharmaceutical ingredient and a biodegradable material, wherein the biodegradable material comprises PLGA (poly(lactic-co-glycolic acid)), PLA (polylactic acid) and PCL (polycaprolactone).

In still another aspect, the present disclosure relates to a process for preparing an ophthalmic mesh-like formulation, comprising

dissolving a biodegradable material and a pharmaceutically acceptable excipient in a solvent to give a mixture;

forming a mesh-like intermediate with the mixture via 3D printing, mold-based hot embossing, mold-based centrifuging, mold-based solvent-casting, mold-based vacuum, injection molding, mold-based photopolymerization, stretching photolithography, solvent-casting, hot melt extrusion, hot molding, or compression molding; and

spraying an active pharmaceutical ingredient on surface of the mesh-like intermediate;

wherein the ophthalmic mesh-like formulation comprises an active pharmaceutical ingredient, a biodegradable material and a pharmaceutically acceptable excipient and wherein the ophthalmic mesh-like formulation has a thickness of not more than 2 mm, a long diameter of not more than 22 mm, and a short diameter of not more than 4 mm.

In yet another aspect, the present disclosure relates to a process for preparing an ophthalmic mesh-like formulation, comprising

dissolving or dispersing an active pharmaceutical ingredient, a biodegradable material and a pharmaceutically acceptable excipient in a solvent to give a mixture; and

forming the mesh-like formulation with the mixture via 3D printing, mold-based hot embossing, mold-based centrifuging, mold-based solvent-casting, mold-based vacuum, injection molding, mold-based photopolymerization, stretching photolithography, solvent-casting, hot melt extrusion, hot molding, or compression molding;

wherein the ophthalmic mesh-like formulation comprises an active pharmaceutical ingredient, a biodegradable material and a pharmaceutically acceptable excipient and wherein the ophthalmic mesh-like formulation has a thickness of not more than 2 mm, a long diameter of not more than 22 mm, and a short diameter of not more than 4 mm.

In still another aspect, the present disclosure relates to a process for preparing an ophthalmic mesh-like formulation, comprising

dissolving an active pharmaceutical ingredient and a biodegradable material in a solvent to give a mixture; and

forming the ophthalmic mesh-like formulation with the mixture via solvent-casting,

wherein the ophthalmic mesh-like formulation comprises an active pharmaceutical ingredient and a biodegradable material, and the biodegradable material comprises PLGA (poly(lactic-co-glycolic acid)) and PLA (polylactic acid).

In yet another aspect, the present disclosure relates to a process for preparing an ophthalmic mesh-like formulation, comprising

dissolving an active pharmaceutical ingredient and a biodegradable material in a solvent to give a mixture; and

forming the ophthalmic mesh-like formulation with the mixture via solvent-casting,

wherein the ophthalmic mesh-like formulation comprises an active pharmaceutical ingredient and a biodegradable material, and the biodegradable material comprises PLGA (poly(lactic-co-glycolic acid)), PLA (polylactic acid) and PCL (polycaprolactone).

In still another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation comprises an active pharmaceutical ingredient, a biodegradable material and a pharmaceutically acceptable excipient and wherein the ophthalmic mesh-like formulation has a thickness of not more than 2 mm, a long diameter of not more than 22 mm, and a short diameter of not more than 4 mm.

In yet another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation comprises an active pharmaceutical ingredient and a biodegradable material, and the biodegradable material comprises PLGA (poly(lactic-co-glycolic acid)) and PLA (polylactic acid).

In still another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation comprises an active pharmaceutical ingredient and a biodegradable material, and the biodegradable material comprises PLGA (poly(lactic-co-glycolic acid)), PLA (polylactic acid) and PCL (polycaprolactone).

In yet another aspect, the present disclosure relates to an ophthalmic implant, comprising an active pharmaceutical ingredient, a biodegradable material and a pharmaceutically acceptable excipient, wherein the ophthalmic implant formulation has a length of not more than 22 mm and a diameter of not more than 2 mm.

In still another aspect, the present disclosure relates to an ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

In yet another aspect, the present disclosure relates to a process for preparing an ophthalmic implant, comprising

preparing the ophthalmic implant via by 3D printing, solvent-casting, hot melt extrusion, hot molding, or compression molding,

wherein the ophthalmic implant comprises an active pharmaceutical ingredient, a biodegradable material and a pharmaceutically acceptable excipient and wherein the ophthalmic implant has a length of not more than 22 mm and a diameter of not more than 2 mm.

In still another aspect, the present disclosure relates to a process for preparing an ophthalmic implant, comprising preparing the ophthalmic implant via hot melt extrusion,

wherein the ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

In yet another aspect, the present disclosure relates to a method for treating and preventing oculopathy, comprising

inserting an ophthalmic implant in a subconjunctival plane at or just superior to a superior tarsal border, or inserting an ophthalmic implant between orbicularis oculi muscle and levator aponeurosis,

wherein the ophthalmic implant comprises an active pharmaceutical ingredient, a biodegradable material and a pharmaceutically acceptable excipient and wherein the ophthalmic implant has a length of not more than 22 mm and a diameter of not more than 2 mm.

In still another aspect, the present disclosure relates to a method for treating and preventing oculopathy, comprising

inserting an ophthalmic implant in a subconjunctival plane at or just superior to a superior tarsal border, or inserting an ophthalmic implant between orbicularis oculi muscle and levator aponeurosis,

wherein the ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

### DETAILED DESCRIPTION

In the following description, certain specific details are included to provide a thorough understanding for various disclosed embodiments. One skilled in the relevant art, however, will recognize that the embodiments may be practiced without one or more these specific details, or with other methods, components, materials, etc.

Unless the context required otherwise, throughout the specification and claims which follows, the term “comprise” and variations thereof, such as “comprises” and “comprising” are to be construed in an open, inclusive sense, which is as “include, but not limited to”.

Reference throughout this specification to “one embodiment”, or “an embodiment”, or “in another embodiment”, or “in some embodiments” means that a particular referent feature, structure or characteristic described in connection with the embodiments is included in at least one embodiment. Therefore, the appearance of the phrases “in one embodiment”, or “in the embodiment”, or “in another embodiment”, or “in some embodiments” in various places throughout this specification are not necessarily all referring to the same embodiment. Moreover, the particular features, structures or characteristics may be combined in any suitable manner in one or more embodiments.

It should be noted that, as used in this specification and the appended claims, the singular forms “a”, “an” and “the” include plural referents unless the context clearly stated otherwise. Therefore, for example, a reaction comprising “a pharmaceutically acceptable excipient” comprises one pharmaceutically acceptable excipient, two or more pharmaceutically acceptable excipients.

In one aspect, the present disclosure relates to an ophthalmic mesh-like formulation, comprising an active pharmaceutical ingredient, a biodegradable material and a pharmaceutically acceptable excipient, wherein the mesh-like formulation has a thickness of not more than 2 mm, a long diameter of not more than 22 mm, and a short diameter of not more than 4 mm.

The exemplary shapes of the ophthalmic mesh-like formulation that can be used in the present disclosure include, but not limited to, round shape, oval shape, square shape and rectangle shape.

The exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

The exemplary active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

In some embodiments of the present disclosure, the ophthalmic mesh-like formulation can be porous.

In some embodiments of the present disclosure, the ophthalmic mesh-like formulation can have variable stiffness/flexibility.

In some embodiments of the present disclosure, the ophthalmic mesh-like formulation can be sustained-released in vivo in one week.

In some embodiments of the present disclosure, the ophthalmic mesh-like formulation can be sustained-released in vivo in two weeks.

In some embodiments of the present disclosure, the ophthalmic mesh-like formulation can be sustained-released in vivo in one to three months. In some embodiments of the present disclosure, the ophthalmic mesh-like formulation can be sustained-released in vivo in six months.

In some embodiments of the present disclosure, the ophthalmic mesh-like formulation can be sustained-released in vivo in more than six months.

In another aspect, the present disclosure relates to an ophthalmic mesh-like formulation, comprising an active pharmaceutical ingredient and a biodegradable material, wherein the biodegradable material comprises PLGA (poly(lactic-co-glycolic acid)) and PLA (polylactic acid).

In some embodiments of the present disclosure, a weight ratio of PLGA (poly(lactic-co-glycolic acid)) to PLA (polylactic acid) is about 1:1 to 13:1.

In some embodiments of the present disclosure, a weight ratio of PLGA (poly(lactic-co-glycolic acid)) to PLA (polylactic acid) is about 1:1 to 10:1.

In some embodiments of the present disclosure, a weight ratio of PLGA (poly(lactic-co-glycolic acid)) to PLA (polylactic acid) is about 1:1 to 8:1.

In some embodiments of the present disclosure, a weight ratio of PLGA (poly(lactic-co-glycolic acid)) to PLA (polylactic acid) is about 3:1 to 6:1.

In some embodiments of the present disclosure, a weight ratio of PLA (polylactic acid) to the ophthalmic mesh-like formulation is about 5% to 50%.

In some embodiments of the present disclosure, a weight ratio of PLA (polylactic acid) to the ophthalmic mesh-like formulation is about 10% to 50%. In some embodiments of the present disclosure, a weight ratio of PLA (polylactic acid) to the ophthalmic mesh-like formulation is about 15% to 50%.

In some embodiments of the present disclosure, a weight ratio of PLA (polylactic acid) to the ophthalmic mesh-like formulation is about 20% to 50%.

In some embodiments of the present disclosure, a weight ratio of PLA (polylactic acid) to the ophthalmic mesh-like formulation is about 25% to 50%.

In some embodiments of the present disclosure, a weight ratio of PLA (polylactic acid) to the ophthalmic mesh-like formulation is about 30% to 50%.

In some embodiments of the present disclosure, a weight ratio of PLA (polylactic acid) to the ophthalmic mesh-like formulation is about 35% to 50%. In some embodiments of the present disclosure, a weight ratio of PLA (polylactic acid) to the ophthalmic mesh-like formulation is about 40% to 50%.

In some embodiments of the present disclosure, a weight ratio of PLA (polylactic acid) to the ophthalmic mesh-like formulation is about 45% to 50%.

In some embodiments of the present disclosure, a weight ratio of PLGA (poly(lactic-co-glycolic acid)) to the ophthalmic mesh-like formulation is about 40% to 76%.

In some embodiments of the present disclosure, a weight ratio of PLGA (poly(lactic-co-glycolic acid)) to the ophthalmic mesh-like formulation is about 42% to 76%. In some embodiments of the present disclosure, a weight ratio of PLGA (poly(lactic-co-glycolic acid)) to the ophthalmic mesh-like formulation is about 44% to 76%.

In some embodiments of the present disclosure, a weight ratio of PLGA (poly(lactic-co-glycolic acid)) to the ophthalmic mesh-like formulation is about 46% to 76%.

In some embodiments of the present disclosure, a weight ratio of PLGA (poly(lactic-co-glycolic acid)) to the ophthalmic mesh-like formulation is about 48% to 76%.

In some embodiments of the present disclosure, a weight ratio of PLGA (poly(lactic-co-glycolic acid)) to the ophthalmic mesh-like formulation is about 50% to 76%.

In some embodiments of the present disclosure, a weight ratio of PLGA (poly(lactic-co-glycolic acid)) to the ophthalmic mesh-like formulation is about 52% to 76%.

In some embodiments of the present disclosure, a weight ratio of PLGA (poly(lactic-co-glycolic acid)) to the ophthalmic mesh-like formulation is about 54% to 76%.

In some embodiments of the present disclosure, a weight ratio of PLGA (poly(lactic-co-glycolic acid)) to the ophthalmic mesh-like formulation is about 56% to 76%.

In some embodiments of the present disclosure, a weight ratio of PLGA (poly(lactic-co-glycolic acid)) to the ophthalmic mesh-like formulation is about 58% to 76%.

In some embodiments of the present disclosure, a weight ratio of PLGA (poly(lactic-co-glycolic acid)) to the ophthalmic mesh-like formulation is about 60% to 76%.

In some embodiments of the present disclosure, a weight ratio of PLGA (poly(lactic-co-glycolic acid)) to the ophthalmic mesh-like formulation is about 62% to 76%.

In some embodiments of the present disclosure, a weight ratio of PLGA (poly(lactic-co-glycolic acid)) to the ophthalmic mesh-like formulation is about 64% to 76%.

In some embodiments of the present disclosure, a weight ratio of PLGA (poly(lactic-co-glycolic acid)) to the ophthalmic mesh-like formulation is about 66% to 76%.

In some embodiments of the present disclosure, a weight ratio of PLGA (poly(lactic-co-glycolic acid)) to the ophthalmic mesh-like formulation is about 68% to 76%.

In some embodiments of the present disclosure, a weight ratio of PLGA (poly(lactic-co-glycolic acid)) to the ophthalmic mesh-like formulation is about 70% to 76%.

In some embodiments of the present disclosure, a weight ratio of PLGA (poly(lactic-co-glycolic acid)) to the ophthalmic mesh-like formulation is about 72% to 76%.

In some embodiments of the present disclosure, a weight ratio of PLGA (poly(lactic-co-glycolic acid)) to the ophthalmic mesh-like formulation is about 74% to 76%.

In some embodiments of the present disclosure, the biodegradable material further comprises PCL (polycaprolactone).

In some embodiments of the present disclosure, a weight ratio of PLGA (poly(lactic-co-glycolic acid)) to PLA (polylactic acid) to PCL (polycaprolactone) is about 2:1:1 to 13:1:1.

In some embodiments of the present disclosure, a weight ratio of PLGA (poly(lactic-co-glycolic acid)) to PLA (polylactic acid) to PCL (polycaprolactone) is about 4:1:1 to 6:1:1.

In some embodiments of the present disclosure, the mesh-like formulation has a thickness of not more than about 2 mm.

In some embodiments of the present disclosure, the mesh-like formulation has a long diameter of not more than about 22 mm.

In some embodiments of the present disclosure, the mesh-like formulation has a short diameter of not more than about 4 mm.

The exemplary shapes of the ophthalmic mesh-like formulation that can be used in the present disclosure include, but not limited to, round shape, oval shape, square shape and rectangle shape.

The exemplary active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

In some embodiments of the present disclosure, he ophthalmic mesh-like formulation can be porous.

In some embodiments of the present disclosure, the ophthalmic mesh-like formulation can have variable stiffness/flexibility.

In some embodiments of the present disclosure, the ophthalmic mesh-like formulation can be sustained-released in vivo in one week.

In some embodiments of the present disclosure, the ophthalmic mesh-like formulation can be sustained-released in vivo in two weeks.

In some embodiments of the present disclosure, the ophthalmic mesh-like formulation can be sustained-released in vivo in one to three months.

In some embodiments of the present disclosure, the ophthalmic mesh-like formulation can be sustained-released in vivo in six months.

In some embodiments of the present disclosure, the ophthalmic mesh-like formulation can be sustained-released in vivo in more than six months.

In some embodiments of the present disclosure, the ophthalmic mesh-like formulation can have lower level of autohesion such that the ophthalmic mesh-like formulation can readily unfold after being inserted.

In yet another aspect, the present disclosure relates to an ophthalmic mesh-like formulation, comprising an active pharmaceutical ingredient and a biodegradable material, wherein the biodegradable material comprises PLGA (poly(lactic-co-glycolic acid)), PLA (polylactic acid) and PCL (polycaprolactone).

In still another aspect, the present disclosure relates to a process for preparing an ophthalmic mesh-like formulation, comprising

dissolving a biodegradable material and a pharmaceutically acceptable excipient in a solvent to give a mixture;

forming a mesh-like intermediate with the mixture via 3D printing, mold-based hot embossing, mold-based centrifuging, mold-based solvent-casting, mold-based vacuum, mold-based photopolymerization, droplet-born air blowing, stretching photolithography, solvent-casting, hot melt extrusion, hot molding, or compression molding; and

spraying an active pharmaceutical ingredient on surface of the mesh-like intermediate;

wherein the mesh-like formulation has a thickness of not more than 2 mm, a long diameter of not more than 22 mm, and a short diameter of not more than 4 mm.

The exemplary solvents that can be used in the present disclosure include, but not limited to, N-methyl pyrrolidone (NMP), glacial acetic acid, dichloromethane, chloroform, acetone, N,N-Dimethylformamide, tetrahydrofuran and ethyl acetate.

In some embodiments of the present disclosure, the active pharmaceutical ingredient can be in the form of solution or suspension.

The exemplary 3D printing that can be used in the present disclosure includes but is not limited to fused deposition modelling, direct metal laser-sintering, electron beam melting, selective laser sintering, selective laser melting, selective heat sintering, stereo lithography appearance, digital light processing, polyjet, multi-jet printing, continuous liquid interface production, two-photon polymerization, 3DP (three dimensional printing) and gluing, binder jetting, color jet printing, nanoparticle jetting, laminated object manufacturing, laser engineered net shaping, multi-jet fusion, plaster-based 3D printing, laser cladding forming and syringe-pump-based 3D printing.

In yet another aspect, the present disclosure relates to a process for preparing an ophthalmic mesh-like formulation, comprising

dissolving or dispersing an active pharmaceutical ingredient, a biodegradable material and a pharmaceutically acceptable excipient in a solvent to give a mixture; and

forming the mesh-like formulation with the mixture via 3D printing, mold-based hot embossing, mold-based centrifuging, mold-based solvent-casting, mold-based vacuum, mold-based photopolymerization, droplet-born air blowing, stretching photolithography, solvent-casting, hot melt extrusion, hot molding, or compression molding;

wherein the mesh-like formulation has a thickness of not more than 2 mm, a long diameter of not more than 22 mm, and a short diameter of not more than 4 mm.

The exemplary 3D printing that can be used in the present disclosure includes but is not limited to fused deposition modelling, direct metal laser-sintering, electron beam melting, selective laser sintering, selective laser melting, selective heat sintering, stereo lithography appearance, digital light processing, polyjet, multi-jet printing, continuous liquid interface production, two-photon polymerization, 3DP (three dimensional printing) and gluing, binder jetting, color jet printing, nanoparticle jetting, laminated object manufacturing, laser engineered net shaping, multi jet fusion, plaster-based 3D printing, laser cladding forming and syringe-pump-based 3D printing.

In still another aspect, the present disclosure relates to a process for preparing an ophthalmic mesh-like formulation, comprising

dissolving an active pharmaceutical ingredient and a biodegradable material in a solvent to give a mixture; and

forming the ophthalmic mesh-like formulation with the mixture via solvent-casting,

wherein the ophthalmic mesh-like formulation comprises an active pharmaceutical ingredient and a biodegradable material, and the biodegradable material comprises PLGA (poly(lactic-co-glycolic acid)) and PLA (polylactic acid).

The exemplary solvents that can be used in the present disclosure include, but not limited to, N-methyl pyrrolidone (NMP), glacial acetic acid, dichloromethane, chloroform, acetone, N,N-Dimethylformamide, tetrahydrofuran and ethyl acetate.

In yet another aspect, the present disclosure relates to a process for preparing an ophthalmic mesh-like formulation, comprising

dissolving an active pharmaceutical ingredient and a biodegradable material in a solvent to give a mixture; and

forming the ophthalmic mesh-like formulation with the mixture via solvent-casting,

wherein the ophthalmic mesh-like formulation comprises an active pharmaceutical ingredient and a biodegradable material, and the biodegradable material comprises PLGA (poly(lactic-co-glycolic acid)), PLA (polylactic acid) and PCL (polycaprolactone).

The exemplary solvents that can be used in the present disclosure include, but not limited to, N-methyl pyrrolidone (NMP), glacial acetic acid, dichloromethane, chloroform, acetone, N,N-Dimethylformamide, tetrahydrofuran and ethyl acetate.

In still another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation has a thickness of not more than 2 mm, a long diameter of not more than 22 mm, and a short diameter of not more than 4 mm.

The exemplary oculopathy that can be treated or prevented by the method of the present disclosure includes but is not limited to ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the method comprises injecting a subcutaneous local anesthetic in an upper eyelid.

In some embodiments of the present disclosure, the method comprises injecting a subconjunctival anesthetic in the plane just under the palpebral conjunctiva.

In some embodiments of the present disclosure, the method comprises making a small buttonhole in a lateral palpebral conjunctiva just superior to a superior tarsal border.

In some embodiments of the present disclosure, the method comprises applying an ophthalmic antibiotic ointment or eyedrop for 3-4 days.

In some embodiments of the present disclosure, method for treating and preventing oculopathy, comprising

applying a drop of ophthalmic topical anesthetic to an affected eye of subject in need thereof;

injecting a subcutaneous local anesthetic in an upper eyelid;

cleaning the surgical area in a standard, sterile, oculoplastic and ophthalmic manner with betadine® swabs;

everting the upper eyelid to expose palpebral conjunctiva;

injecting a subconjunctival anesthetic in a plane just under the palpebral conjunctiva;

making a small buttonhole in the lateral palpebral conjunctiva just superior to a superior tarsal border;

creating a small pocket in a plane between palpebral conjunctiva and Muller's muscle for inserting an ophthalmic mesh-like formulation via blunt dissection;

inserting the ophthalmic mesh-like formulation by an inserter;

performing an appropriate hemostasis;

checking the surgical area to ensure no exposure of the ophthalmic mesh-like formulation;

returning the eyelid to its normal anatomic position;

cleaning the surgical area with sterile saline; and

applying an ophthalmic antibiotic ointment or eyedrop is applied for 3-4 days.

The treatment methods and ophthalmic mesh-like formulations described in the present disclosure have the advantages of targeted, local administration and minimization of systemic side effects.

In yet another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation comprises an active pharmaceutical ingredient and a biodegradable material, and the biodegradable material comprises PLGA (poly(lactic-co-glycolic acid)) and PLA (polylactic acid).

In still another aspect, the present disclosure relates to a method for treating and preventing oculopathy, comprising

creating a small pocket in a plane between palpebral conjunctiva and Muller's muscle of an affected eye of a subject in need thereof; and

inserting an ophthalmic mesh-like formulation into the small pocket;

wherein the ophthalmic mesh-like formulation comprises an active pharmaceutical ingredient and a biodegradable material, and the biodegradable material comprises PLGA (poly(lactic-co-glycolic acid)), PLA (polylactic acid) and PCL (polycaprolactone).

The exemplary oculopathy that can be treated or prevented by the method of the present disclosure includes but is not limited to ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the method comprises injecting a subcutaneous local anesthetic in an upper eyelid.

In some embodiments of the present disclosure, the method comprises injecting a subconjunctival anesthetic in the plane just under the palpebral conjunctiva.

In some embodiments of the present disclosure, the method comprises making a small buttonhole in a lateral palpebral conjunctiva just superior to a superior tarsal border.

In some embodiments of the present disclosure, the method comprises applying an ophthalmic antibiotic ointment or eyedrop for 3-4 days.

In some embodiments of the present disclosure, method for treating and preventing oculopathy, comprising

applying a drop of ophthalmic topical anesthetic to an affected eye of subject in need thereof;

injecting a subcutaneous local anesthetic in an upper eyelid;

cleaning the surgical area in a standard, sterile, oculoplastic and ophthalmic manner with betadine swabs;

everting the upper eyelid to expose palpebral conjunctiva;

injecting a subconjunctival anesthetic in a plane just under the palpebral conjunctiva;

making a small buttonhole in the lateral palpebral conjunctiva just superior to a superior tarsal border;

creating a small pocket in a plane between palpebral conjunctiva and Muller's muscle for inserting the ophthalmic mesh-like formulation via blunt dissection;

inserting the ophthalmic mesh-like formulation by an inserter;

performing an appropriate hemostasis;

checking the surgical area to ensure no exposure of the ophthalmic mesh-like formulation;

returning the eyelid to its normal anatomic position;

cleaning the surgical area with sterile saline; and

applying an ophthalmic antibiotic ointment or eyedrop is applied for 3-4 days.

In some embodiments of the present disclosure, the anesthetic can contain 1:100,000 parts of epinephrine.

In some embodiments of the present disclosure, the anesthetic can contain 1% lidocaine with 1:100,000 epinephrine.

In some embodiments of the present disclosure, blunt dissection can help avoid lacerating the blood vessels.

In some embodiments of the present disclosure, the ophthalmic mesh-like formulation can be inserted by a syringe.

The treatment methods and ophthalmic mesh-like formulations described in the present disclosure have the advantages of targeted, local administration and minimization of systemic side effects.

In yet another aspect, the present disclosure relates to an ophthalmic implant, comprising an active pharmaceutical ingredient, a biodegradable material and a pharmaceutically acceptable excipient, wherein the ophthalmic implant formulation has a length of not more than 22 mm and a diameter of not more than 2 mm.

The exemplary shapes of the ophthalmic implant that can be used in the present disclosure include, but not limited to, rod-like shape, cannula-like shape, round shape, oval shape, square shape and rectangle shape.

The exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

The exemplary active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

In some embodiments of the present disclosure, the ophthalmic implant can have variable stiffness/flexibility.

In some embodiments of the present disclosure, the ophthalmic implant can be sustained-released in vivo in one week.

In some embodiments of the present disclosure, the ophthalmic implant can be sustained-released in vivo in two weeks.

In some embodiments of the present disclosure, the ophthalmic implant can be sustained-released in vivo in one to three months.

In some embodiments of the present disclosure, the ophthalmic implant can be sustained-released in vivo in six months.

In some embodiments of the present disclosure, the ophthalmic implant can be sustained-released in vivo in more than six months.

In still another aspect, the present disclosure relates to an ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

In some embodiments of the present disclosure, the ophthalmic implant has a length of not more than about 22 mm.

In some embodiments of the present disclosure, the ophthalmic implant has a diameter of not more than about 2 mm.

The exemplary shapes of the ophthalmic implant that can be used in the present disclosure include, but not limited to, rod-like shape, cannula-like shape, round shape, oval shape, square shape and rectangle shape.

The exemplary active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

In some embodiments of the present disclosure, the ophthalmic implant can have variable stiffness/flexibility.

In some embodiments of the present disclosure, the ophthalmic implant can be sustained-released in vivo in one week.

In some embodiments of the present disclosure, the ophthalmic implant can be sustained-released in vivo in two weeks.

In some embodiments of the present disclosure, the ophthalmic implant can be sustained-released in vivo in one to three months.

In some embodiments of the present disclosure, the ophthalmic implant can be sustained-released in vivo in six months.

In some embodiments of the present disclosure, the ophthalmic implant can be sustained-released in vivo in more than six months.

In yet another aspect, the present disclosure relates to a process for preparing the ophthalmic implant, comprising

preparing the ophthalmic implant via by 3D printing, solvent-casting, hot melt extrusion, hot molding, or compression molding,

wherein the ophthalmic implant comprises an active pharmaceutical ingredient, a biodegradable material and a pharmaceutically acceptable excipient and wherein the ophthalmic implant has a length of not more than 22 mm and a diameter of not more than 2 mm.

The exemplary 3D printing that can be used in the present disclosure includes but is not limited to fused deposition modelling, direct metal laser-sintering, electron beam melting, selective laser sintering, selective laser melting, selective heat sintering, stereo lithography appearance, digital light processing, polyjet, multi-jet printing, continuous liquid interface production, two-photon polymerization, 3DP (three dimensional printing) and gluing, binder jetting, color jet printing, nanoparticle jetting, laminated object manufacturing, laser engineered net shaping, multi-jet fusion, plaster-based 3D printing, laser cladding forming and syringe-pump-based 3D printing.

In still another aspect, the present disclosure relates to a process for preparing an ophthalmic implant, comprising preparing the ophthalmic implant via hot melt extrusion,

wherein the ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

In yet another aspect, the present disclosure relates to a method for treating and preventing oculopathy, comprising

inserting an ophthalmic implant in a subconjunctival plane at or just superior to a superior tarsal border, or inserting an ophthalmic implant between orbicularis oculi muscle and levator aponeurosis,

wherein the ophthalmic implant comprises an active pharmaceutical ingredient, a biodegradable material and a pharmaceutically acceptable excipient and

wherein the ophthalmic implant has a length of not more than 22 mm and a diameter of not more than 2 mm.

The exemplary oculopathy that can be treated or prevented by the method of the present disclosure includes but is not limited to ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the ophthalmic implant is inserted between orbicularis oculi muscle and levator aponeurosis via posterior approach.

In some embodiments of the present disclosure, the ophthalmic implant is inserted between orbicularis oculi muscle and levator aponeurosis via external approach through subcutaneous injection.

In some embodiments of the present disclosure, the method comprises prior to everting an upper eyelid to expose a palpebral conjunctiva cleaning the surgical area in a standard, sterile, oculoplastic and ophthalmic manner with betadine® swabs.

In some embodiments of the present disclosure, the method comprises after inserting the ophthalmic implant cleaning the surgical area with sterile saline.

In some embodiments of the present disclosure, the method comprises applying a topical antibiotic coverage to the eye for 3-4 days.

In some embodiments of the present disclosure, the method comprises returning the eyelid to its normal anatomic position.

In some embodiments of the present disclosure, the method for treating and preventing oculopathy, comprising

applying a drop of ophthalmic topical anesthetic to an affected eye of a subject in need thereof;

cleaning the surgical area in a standard, sterile, oculoplastic and ophthalmic manner with betadine® swabs;

everting an upper eyelid to expose a palpebral conjunctiva;

inserting an ophthalmic implant in a subconjunctival plane at or just superior to a superior tarsal border, or inserting an ophthalmic implant between orbicularis oculi muscle and levator aponeurosis via posterior approach or via external approach through subcutaneous injection;

cleaning the surgical area with sterile saline;

applying a topical antibiotic coverage to the eye for 3-4 days; and

returning the eyelid to its normal anatomic position.

The treatment methods and ophthalmic implant described in the present disclosure have the advantages of targeted, local administration and minimization of systemic side effects.

In still another aspect, the present disclosure relates to a method for treating and preventing oculopathy, comprising

inserting an ophthalmic implant in a subconjunctival plane at or just superior to a superior tarsal border, or inserting an ophthalmic implant between orbicularis oculi muscle and levator aponeurosis,

wherein the ophthalmic implant, comprising an active pharmaceutical ingredient, a biodegradable material and poly(ethylene oxide), wherein the biodegradable material is selected from the group consisting of PLGA (poly (lactic-co-glycolic acid)), PLA (polylactic acid) and a mixture thereof.

The exemplary oculopathy that can be treated or prevented by the method of the present disclosure includes but is not limited to ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the ophthalmic implant is inserted between orbicularis oculi muscle and levator aponeurosis via posterior approach.

In some embodiments of the present disclosure, the ophthalmic implant is inserted between orbicularis oculi muscle and levator aponeurosis via external approach through subcutaneous injection.

In some embodiments of the present disclosure, the method comprises prior to everting an upper eyelid to expose a palpebral conjunctiva cleaning the surgical area in a standard, sterile, oculoplastic and ophthalmic manner with betadine® swabs.

In some embodiments of the present disclosure, the method comprises after inserting the ophthalmic implant cleaning the surgical area with sterile saline.

In some embodiments of the present disclosure, the method comprises applying a topical antibiotic coverage to the eye for 3-4 days.

In some embodiments of the present disclosure, the method comprises returning the eyelid to its normal anatomic position.

In some embodiments of the present disclosure, the method for treating and preventing oculopathy, comprising applying a drop of ophthalmic topical anesthetic to an affected eye of a subject in need thereof;

cleaning the surgical area in a standard, sterile, oculoplastic and ophthalmic manner with betadine® swabs;

everting an upper eyelid to expose a palpebral conjunctiva;

inserting an ophthalmic implant in a subconjunctival plane at or just superior to a superior tarsal border, or inserting an ophthalmic implant between orbicularis oculi muscle and levator aponeurosis via posterior approach or via external approach through subcutaneous injection;

cleaning the surgical area with sterile saline;

applying a topical antibiotic coverage to the eye for 3-4 days; and

returning the eyelid to its normal anatomic position.

The treatment methods and ophthalmic implant described in the present disclosure have the advantages of targeted, local administration and minimization of systemic side effects.

### EXAMPLES

Although anyone skilled in the art is capable of preparing the formulations of the present disclosure according to the general techniques disclosed above, more specific details on synthetic techniques for formulations of the present disclosure are provided elsewhere in this specification for convenience. Again, all reagents and reaction conditions employed in synthesis are known to those skilled in the art and are available from ordinary commercial sources.

### Materials and Experiment Equipment:

Neostigmine bromide: Hubei Guangao Biotechnology Co., Ltd./GA20181205

Trichloromethane: Guangzhou Chemical Reagent Factory/20180706

PLGA: Jinan Daigang Biomaterial Co., Ltd./20181112804 & 2019050711

PCL: Jinan Daigang Biomaterial Co., Ltd./2018101210 & 2019042306

PLA: Jinan Daigang Biomaterial Co., Ltd./2018120605 & 2019031212

Poly(ethylene oxide): Shanghai Colorcon Coating Technology Co.Ltd./D682H5APL6

Hot melt extruder: Thermo Fisher Scientific/Pharma mini HME II

Balance with a precision of 1/10,000: METTLER TOLEDO Group/XS204

### Example 1

Preparation of drug-loading solution:

0.1 g neostigmine bromide, 0.5 g PLA (polylactic acid) were added into

10 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 2

Preparation of drug-loading solution:

0.1 g neostigmine bromide, 0.25 g PLA (polylactic acid) and 0.25 g PLGA (poly (lactic-co-glycolic acid)) were added into 10 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 3

Preparation of drug-loading solution:

0.05 g neostigmine bromide, 0.063 g PLA (polylactic acid) and 0.187 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 4

Preparation of drug-loading solution:

0.05 g neostigmine bromide, 0.036 g PLA (polylactic acid) and 0.214 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 5

Preparation of drug-loading solution:

0.05 g neostigmine bromide, 0.028 g PLA (polylactic acid) and 0.222 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 6

Preparation of drug-loading solution:

0.05 g neostigmine bromide, 0.023 g PLA (polylactic acid) and 0.227 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 7

Preparation of drug-loading solution:

0.1 g neostigmine bromide, 0.5 g PLGA (poly (lactic-co-glycolic acid)) were added into 10 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 8

Preparation of drug-loading solution:

0.05 g neostigmine bromide, 0.064 g PLA (polylactic acid), 0.064 g PCL (polycaprolactone) and 0.124 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 9

Preparation of drug-loading solution:

0.05 g neostigmine bromide, 0.042 g PLA (polylactic acid), 0.042 g PCL (polycaprolactone) and 0.167 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 10

Preparation of drug-loading solution:

0.05 g neostigmine bromide, 0.031 g PLA (polylactic acid), 0.031 g PCL (polycaprolactone) and 0.188 g PLGA (poly (lactic-co-glycolic acid)) were added into 5 mL trichloromethane solution to get solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 11

Preparation of drug-loading solution:

0.05 g neostigmine bromide, 0.017 g PLA (polylactic acid), 0.017 g PCL (polycaprolactone) and 0.216 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 12

Preparation of drug-loading solution:

0.05 g neostigmine bromide, 0.013 g PLA (polylactic acid), 0.013 g PCL (polycaprolactone) and 0.225 g PLGA (poly (lactic-co-glycolic acid)) were added into 5 mL trichloromethane solution to give solution A.

Preparation of mesh-like formulation:

Solution A was poured onto a polypropylene cap of 50 mL centrifugal tube. The solvent was evaporated. The drug-loading cap was dried at the room temperature for 12 hours. The cap was removed to give the mesh-like formulation.

### Example 13

Tests on flexibility of mesh-like formulation

The mesh-like formulation with a diameter of 2.5 cm was used. The mesh-like formulation was put on a table. Half area of the formulation (area A) was placed outside the edge of the table. Polytetrafluoroethylene/silica gel gaskets were added gradually onto the area A until area A cannot bear the weight of polytetrafluoroethylene/silica gel gaskets. Polytetrafluoroethylene/silica gel gaskets were collected and weighed to evaluate the flexibility of the mesh-like formulation (Table 1 and Table 2).

TABLE 1

| Examples | PLGA:PLA | weight/mg |
|---|---|---|
| 1 | 0:1 | 357.52 |
| 2 | 1:1 | 258.45 |
| 3 | 3:1 | 169.574 |
| 4 | 6:1 | 121.735 |
| 5 | 8:1 | 114.575 |
| 6 | 10:1 | 45.572 |
| 7 | 1:0 | 19.273 |

TABLE 2

| Examples | PLGA:PLA:PCL | Reading/mg |
|---|---|---|
| 8 | 2:1:1 | 208.545 |
| 9 | 4:1:1 | 185.467 |
| 10 | 6:1:1 | 168.543 |
| 11 | 13:1:1 | 87.543 |
| 12 | 18:1:1 | 22.577 |

### Example 14

Tests on autohesion of mesh-like formulation

Two mesh-like formulations (films) with a size of 1.2 cm×1.0 cm were pressed and staggered to adhere together and were put on a balance with a precision of 1/10,000. One of the two formulations was pressed with a weight, while the other one was picked up slowly with tweezers. The maximum variation value of the balance was recorded and used to evaluate the autohesion of the mesh-like formulation (Table 3 and Table 4).

TABLE 3
Autohesion of mesh-like formulation

| | Maximum variation value of |
|---|---|
| Examples | balance (mg) |
| 1 (PLGA:PLA = 0:1) | * |
| 2 (PLGA:PLA = 1:1) | * |
| 3 (PLGA:PLA = 3:1) | −187.985 |
| 4 (PLGA:PLA = 6:1) | −317.84 |
| 5 (PLGA:PLA = 8:1) | −400.625 |
| 6 (PLGA:PLA = 10:1) | −600.69 |
| 7 (PLGA:PLA = 1:0) | −757.94 |

TABLE 4
Autohesion of mesh-like formulation

| | Maximum variation value of |
|---|---|
| Examples | balance (mg) |
| 8 (PLGA:PLA:PCL = 2:1:1) | * |
| 9 (PLGA:PLA:PCL = 4:1:1) | * |
| 10 (PLGA:PLA:PCL = 6:1:1) | −47.935 |
| 11 (PLGA:PLA:PCL = 13:1:1) | −280.33 |
| 12 (PLGA:PLA:PCL = 18:1:1) | −684.31 |

### Example 15

0.4 g neostigmine bromide, 2.00 g PLGA (poly (lactic-co-glycolic acid)), and 2.00 g poly(ethylene oxide) were weighed and then mixed. The mixture was put into a hot melt extruder and extruded to give thermoplastic filaments. The temperatures of extruder zone 1 and zone 2 were 170° C. and 110° C., respectively. The speed of extruder motor was 10 to 15 rpm. The diameter of the thermoplastic filament was 1.2 to 1.7 mm.

### Example 16

0.4 g neostigmine bromide, 2.00 g PCL (polycaprolactone), and 2.00 g poly(ethylene oxide) were weighed and then mixed. The mixture was put into a hot melt extruder and extruded to give thermoplastic filaments. The temperatures of extruder zone 1 and zone 2 were 185° C. and 165° C., respectively. The speed of extruder motor was 10 to 15 rpm. The diameter of the thermoplastic filament was 1.2 to 1.7 mm.

### Example 17

0.4 g neostigmine bromide, 2.00 g PLA (polylactic acid), and 2.00 g poly(ethylene oxide) were weighed and then mixed. The mixture was put into a hot melt extruder and extruded to give thermoplastic filaments. The temperatures of extruder zone 1 and zone 2 were 190° C. and 165° C., respectively. The speed of extruder motor was 10 to 15 rpm. The diameter of the thermoplastic filament was 1.2 to 1.7 mm.

### Example 18

Tests on flexibility of implant

The implant was folded in half, and then unfolded, and folded again in opposite direction and then unfolded. The above steps were repeated until the implant ruptured. Times of folds were recorded to evaluate the flexibility of the implant (Table 5).

TABLE 5

| Examples | Times of folds |
|---|---|
| 15 | 20 |
| 16 | 0 |
| 17 | 12 |

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

What is claimed is:

1. An ophthalmic mesh-like formulation, comprising an active pharmaceutical ingredient and a biodegradable material, wherein the biodegradable material comprises PLGA (poly(lactic-co-glycolic acid)) and PLA (polylactic acid).

2. The ophthalmic mesh-like formulation of claim 1, wherein a weight ratio of PLGA (poly(lactic-co-glycolic acid)) to PLA (polylactic acid) is 1:1 to 13:1, preferably is 1:1 to 10:1.

3. The ophthalmic mesh-like formulation of claim 1, wherein the biodegradable material further comprises PCL (polycaprolactone).

4. The ophthalmic mesh-like formulation of claim 3, wherein a weight ratio of PLGA (poly(lactic-co-glycolic acid)) to PLA (polylactic acid) to PCL (polycaprolactone) is 2:1:1 to 13:1:1.

5. The ophthalmic mesh-like formulation of claim 1, wherein the mesh-like formulation has a thickness of not more than 2 mm.

6. The ophthalmic mesh-like formulation of claim 1, wherein the mesh-like formulation has a long diameter of not more than 22 mm.

7. The ophthalmic mesh-like formulation of claim 1, wherein the mesh-like formulation has a short diameter of not more than 4 mm.

8. The ophthalmic mesh-like formulation of claim 1, wherein the shape of the ophthalmic mesh-like formulation is round, oval, square or rectangle.

9. The ophthalmic mesh-like formulation of claim 1, wherein the active pharmaceutical ingredient is selected from the group consisting of neostigmine bromide, neostigmine, pyridostigmine, edrophonium chloride, ambenonium chloride, physostigmine, demecarium bromide and galantamine.

10. The ophthalmic mesh-like formulation of claim 1, wherein the ophthalmic mesh-like formulation is porous.

11. The ophthalmic mesh-like formulation of claim 1, wherein the ophthalmic mesh-like formulation has variable stiffness/flexibility.

12. The ophthalmic mesh-like formulation of claim 1, wherein the ophthalmic mesh-like formulation is sustained-released in vivo in one week, two weeks, one to three months, six months or longer.

13. A process for preparing an ophthalmic mesh-like formulation, comprising
dissolving an active pharmaceutical ingredient and a biodegradable material in a solvent to give a mixture; and
forming the ophthalmic mesh-like formulation with the mixture via solvent-casting,
wherein the ophthalmic mesh-like formulation comprises an active pharmaceutical ingredient and a biodegradable material, and the biodegradable material comprises PLGA (poly(lactic-co-glycolic acid)) and PLA (polylactic acid).

14. The process of claim 13, wherein the solvent is selected from the group consisting of N-methyl pyrrolidone (NMP), chloroform, acetone, N,N-Dimethylformamide, tetrahydrofuran, ethyl acetate and a mixture thereof.

15. A method for treating and preventing oculopathy, comprising
creating a small pocket in a plane between palpebral conjunctiva and Muller's muscle of an affected eye of a subject in need thereof and
inserting an ophthalmic mesh-like formulation into the small pocket,
wherein the ophthalmic mesh-like formulation comprises an active pharmaceutical ingredient and a biodegradable material, and the biodegradable material comprises PLGA (poly(lactic-co-glycolic acid)) and PLA (polylactic acid).

16. The method of claim 15, wherein the oculopathy is selected from the group consisting of ocular myasthenia gravis (OMG), blepharospasm, dermatolysis palpebrarum, involutional, myogenic, neurogenic, and congenital ptosis, trichiasis and eyelid tumors.

17. The method of claim 15, wherein the method comprises injecting a subconjunctival anesthetic in the plane just under the palpebral conjunctiva.

18. The method of claim 15, wherein the method comprises making a small buttonhole in the lateral palpebral conjunctiva just superior to the superior tarsal border.

19. The method of claim 17, wherein the anesthetic contains 1:100,000 parts of epinephrine.

20. The method of claim 15, wherein the ophthalmic mesh-like formulation is inserted by a syringe.
